(19) United States
(12) Patent Application Publication
(10) Pub. No.: US 2024/0299346 A1
(43) Pub. Date: Sep. 12, 2024

(54) DIETARY SUPPLEMENT BEADS

(71) Applicant: Phi Health Sciences, Inc., Salt Lake City, UT (US)

(72) Inventors: John-Paul Rowland ANDERSEN, Orem, UT (US); Kirk Roy OUIMET, Highland, UT (US)

(21) Appl. No.: 18/596,418

(22) Filed: Mar. 5, 2024

Related U.S. Application Data
(60) Provisional application No. 63/450,279, filed on Mar. 6, 2023.
(60) Provisional application No. 63/523,575, filed on Jun. 27, 2023.

(51) Int. Cl. A61K 31/375; A61K 9/50; A61K 31/07; A61K 31/593; A61K 31/714; A61K 33/06; A61K 33/26; A61K 33/30; A61K 33/34

(52) U.S. Cl. CPC A61K 31/375; A61K 9/5084; A61K 31/07; A61K 31/593; A61K 31/714; A61K 33/06; A61K 33/26; A61K 33/30; A61K 33/34

(57) ABSTRACT

A dietary supplement bead may include a body comprising iron and vitamin C. A dietary supplement bead may include a microencapsulating material in the body, the microencapsulating material facilitating a sustained release of the iron and the vitamin C simultaneously within a small intestine of a user.

### CROSS-REFERENCE TO RELATED APPLICATIONS

This application claims priority to and the benefit of co-pending U.S. Provisional Patent Application Ser. No. 63/450,279 to John-Paul Andersen, filed on Mar. 6, 2023, and entitled “DIETARY SUPPLEMENT BEADS,” and U.S. Provisional Patent Application Ser. No. 63/523,575 to John-Paul Andersen, filed on Jun. 27, 2023, and entitled “DIETARY SUPPLEMENT BEADS,” both applications the entirety of which are hereby incorporated by reference in their entirety for all intents and purposes by this reference.

### BACKGROUND

Dietary supplements are products intended to supplement the diet and provide additional nutrients or other substances that may be lacking or insufficient in a person's regular diet. These supplements come in many forms, including tablets, capsules, powders, liquids, and bars.

Dietary supplements can include a variety of substances, including vitamins, minerals, amino acids, enzymes, and combinations thereof. These substances can be derived from a range of sources, including plants, animals, and synthetic materials. Vitamins and minerals are nutrients that our bodies use to function properly. Amino acids are the building blocks of proteins and are used for various bodily and metabolic functions. Enzymes are proteins that help catalyze chemical reactions in the body.

Dietary supplements are often formed into one or more pills. For example, one or more vitamins, minerals, amino acids, enzymes, or other nutrients may be collected and formed into a pill. An individual may swallow a pill, and the pill may release the nutrients into the body during digestion or otherwise while the pill remains in the body.

### BRIEF SUMMARY

In some aspects, the techniques described herein relate to a dietary supplement bead. The dietary supplement bead includes a body including a first nutrient and a second nutrient. The first nutrient and the second nutrient facilitate complementary uptake of a user. A microencapsulating material is located the body. The microencapsulating material facilitates a sustained release of the first nutrient and the second nutrient simultaneously within an intestine of a user.

In some aspects, the techniques described herein relate to a dietary supplement bead set to be swallowed simultaneously by a user. The dietary supplement bead set includes a first dietary supplement bead. The first dietary supplement bead includes a first body including a first nutrient and a microencapsulating material in the body. The microencapsulating material facilitating a release of the first nutrient at a first time. A second dietary supplement bead includes a second body including a second nutrient. A microencapsulating layer surrounding the body. The microencapsulating layer facilitates a release of the second nutrient at a second time.

In some aspects, the techniques described herein relate to a dietary supplement bead set. The dietary supplement bead set includes a first dietary supplement bead including a first body including a first nutrient and a second nutrient and a first microencapsulating layer surrounding the first body. The first microencapsulating layer facilitates a sustained release of the first nutrient and the second nutrient simultaneously at a first time. A second dietary supplement bead includes a second body including a third nutrient and a second microencapsulating layer surrounding the second body. The second microencapsulating material layer facilitates a release of the third material at a second time different than the first time.

This summary is provided to introduce a selection of concepts that are further described below in the detailed description. This summary is not intended to identify key or essential features of the claimed subject matter, nor is it intended to be used as an aid in limiting the scope of the claimed subject matter.

Additional features and advantages of embodiments of the disclosure will be set forth in the description which follows, and in part will be obvious from the description, or may be learned by the practice of such embodiments. The features and advantages of such embodiments may be realized and obtained by means of the instruments and combinations particularly pointed out in the appended claims. These and other features will become more fully apparent from the following description and appended claims, or may be learned by the practice of such embodiments as set forth hereinafter.

### BRIEF DESCRIPTION OF THE DRAWINGS

In order to describe the manner in which the above-recited and other features of the disclosure can be obtained, a more particular description will be rendered by reference to specific implementations thereof which are illustrated in the appended drawings. For better understanding, the like elements have been designated by like reference numbers throughout the various accompanying figures. While some of the drawings may be schematic or exaggerated representations of concepts, at least some of the drawings may be drawn to scale. Understanding that the drawings depict some example implementations, the implementations will be described and explained with additional specificity and detail through the use of the accompanying drawings in which:

FIG. 1 is a representation of a dietary supplement system, according to at least one embodiment of the present disclosure.

FIG. 2 is a schematic representation of small intestine, according to at least one embodiment of the present disclosure.

FIG. 3 is a representation of a release chart for a first nutrient release line and a second nutrient release line, according to at least one embodiment of the present disclosure.

FIG. 4 is a schematic representation of a dietary supplement bead, according to at least one embodiment of the present disclosure.

FIG. 5 is a schematic representation of a dietary supplement bead, according to at least one embodiment of the present disclosure.

FIG. 6 is a schematic representation of a dietary supplement bead, according to at least one embodiment of the present disclosure.

FIG. 7 is a schematic representation of a dietary supplement bead, according to at least one embodiment of the present disclosure.

FIG. 9 is a representation of a method for manufacturing a dietary supplement bead, according to at least one embodiment of the present disclosure.

FIG. 8 is a schematic representation of a dietary supplement bead, according to at least one embodiment of the present disclosure.

FIG. 10 illustrates a method for manufacturing a plurality of dietary supplement beads, according to at least one embodiment of the present disclosure.

### DETAILED DESCRIPTION

This disclosure generally relates to devices, systems, and methods for dietary supplement beads. In accordance with at least one embodiment of the present disclosure, the dietary supplement beads discussed herein may have a small form factor to improve the case of ingestion. For example, dietary supplement beads may have a small size (e.g., less than 1 mm) and an associated small weight. The small size of the dietary supplement beads may increase the case of ingestion by the user. For example, people that have a sensitive gag reflex, a small esophagus, and/or a swallowing phobia may not easily swallow conventional supplement pills. This may result in the user not receiving essential vitamins and nutrients. The techniques of the present disclosure, in accordance with one or more embodiments, may facilitate the user swallowing the supplement beads without triggering the user's gag reflex and/or getting stuck in his or her throat. This may increase the case of supplement ingestion, thereby increasing the amount of vitamins and nutrients the user may ingest.

In accordance with at least one embodiment of the present disclosure, dietary supplement beads may be manufactured to time the ingestion of one particular nutrient based another nutrient. For example, the dietary supplement beads may be manufactured to facilitate the simultaneous uptake of two nutrients. The two simultaneously absorbed nutrients may be complementary to each other. For example, the two simultaneously absorbed nutrients may facilitate improved uptake of one or the other or both nutrients, thereby increasing the effectiveness of the dietary supplement beads.

In some examples, the dietary supplement beads may be manufactured to facilitate simultaneous ingestion and different uptake periods. For example, two nutrients, which simultaneously absorbed, may conflict and/or prohibit uptake of one, the other, or both of the two nutrients. Manufacturing the dietary supplement beads for different uptake periods may allow for simultaneous ingestion of the dietary supplement beads while increasing the uptake of the identified nutrients.

In some embodiments, a dietary supplement bead set may include multiple dietary supplement beads. The multiple dietary supplement beads may allow the user to receive a dosage or an ingestion quantity of a particular ingredient. In accordance with at least one embodiment of the present disclosure, the multiple dietary supplement beads may include different types of beads having different nutrients. For example, a first subset of beads may include nutrients that are absorbed at a first time and a second subset of beads may include nutrients that are absorbed at a second time. The user may ingest all of the beads simultaneously, or approximately simultaneously (e.g., in multiple batches), thereby increasing the simplicity of the ingestion schedule for the user.

In accordance with at least one embodiment of the present disclosure, the dietary supplement bead may include microencapsulating material. The microencapsulating material may facilitate the sustained release of the two or more nutrients simultaneously in the small intestine. As a specific, non-limiting example, a dietary supplement bead may include iron and vitamin C. The dietary supplement bead may have a small size. The small size of the dietary supplement bead may help a user to swallow the pill. This may help improve consistency in a dietary supplement regimen, which may help to maintain the nutrient levels in the user's body. Simultaneous release of the iron and the vitamin C may help to improve the uptake of the iron. Iron in dietary supplements may be in the form of Fe²⁺, which may have a limited uptake. Vitamin C may help to convert and/or facilitate conversion of the iron to a form that may be more easily absorbed by the body. Thus, simultaneous release of the iron and the vitamin C may help improve the uptake of iron in the body. In some embodiments, sustained release of the iron and the vitamin C may help to improve uptake of the iron and the vitamin C.

As a specific, non-limiting example, and in accordance with at least one embodiment of the present disclosure, a first dietary supplement bead may include magnesium and a second dietary supplement bead may include calcium. The two dietary supplement beads may have different release profiles, thereby allowing them to be taken simultaneously without interference or with reduced interference in the uptake between the magnesium and the calcium. The dietary supplement bead may have a small size. The small size of the dietary supplement bead may help a user to swallow the pill. This may help improve consistency in a dietary supplement regimen, which may help to maintain the nutrient levels in the user's body.

The dietary supplement beads may have an ordered release to release the nutrients at different times. For example, a calcium supplement bead may be configured to be processed in the small intestine first. A magnesium supplement bead may be configured to be processed in the small intestine after the calcium supplement bead is processed. This may help to improve the absorption of both magnesium and/or calcium in the small intestine. Calcium and magnesium are absorbed at the same absorption sites in the small intestine, and therefore may compete for absorption. Further, magnesium and calcium may form complexes when both are present in the small intestine, and the complex may not be absorbed at the absorption site. Thus, when calcium and magnesium are both present at the same time in the small intestine, the competition for absorption sites and/or the formation of the complex may reduce the absorption rate of one or both of the calcium and magnesium.

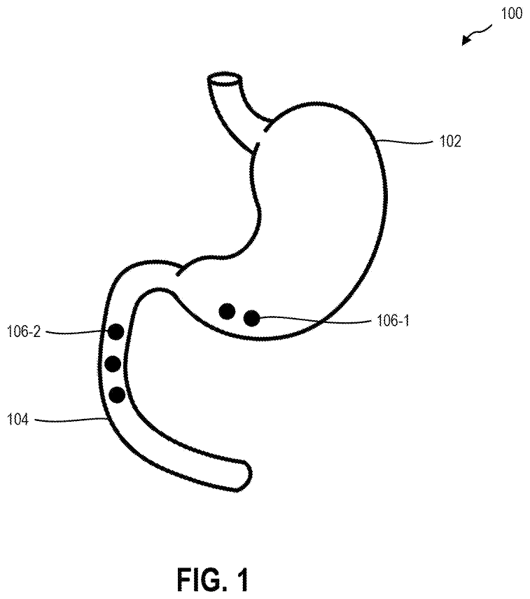

FIG. 1 is a representation of a dietary supplement system 100, according to at least one embodiment of the present disclosure. The dietary supplement system 100 shown includes a schematic representation of a user's stomach 102 and small intestine 104. In the embodiment shown, the user has swallowed multiple dietary supplement beads (collectively 106). The dietary supplement beads 106 may be located at a location within the user's digestive system. For example, a first set of dietary supplement beads 106-1 may be located in the stomach 102 and a second set of dietary supplement beads 106-2 may be located in the small intestine 104.

In the embodiment shown, the user has taken multiple dietary supplement beads 106. Conventionally, a user may take a single dietary supplement pill, or a couple pills. Many people have difficulty swallowing pills. For example, dietary supplement pills may engage a user's gag-reflex, may get caught in the user's esophagus, may be too large for a user's esophagus, or otherwise be difficult for a user to swallow.

In accordance with at least one embodiment of the present disclosure, the dietary supplement beads 106 may have a small size. For example, the dietary supplement beads 106 may have a diameter that is approximately equal to 0.5 mm. This small size may help the user to swallow the dietary supplement beads 106. This may help to improve the user's comfort, increase the consistency of the user taking the dietary supplements, improve the compliance in a supplement system, and otherwise improve the user experience.

Based on their size, each individual dietary supplement bead 106 may not include the desired amount of one or more of the nutrients. To ingest the desired amount of nutrients, the user may swallow multiple dietary supplement beads 106. Because the dietary supplement beads 106 are so small, the user may easily swallow multiple dietary supplement beads 106. To ingest the desired amount of nutrients, the user may ingest an ingestion quantity of the dietary supplement beads 106. In some embodiments, the ingestion quantity may be in a range having an upper value, a lower value, or upper and lower values including any of 2, 3, 4, 5, 10, 15, 20, 25, 30, 40, 50, 75, 100, or any value therebetween. For example, the ingestion quantity may be greater than 2. In another example, the ingestion quantity may be less than 100. In yet other examples, the ingestion quantity may be any value in a range between 2 and 100. In some embodiments, it may be critical that the ingestion quantity is greater than 10 to facilitate easy ingestion of the desired amount of the nutrients in the dietary supplement beads 106.

The dietary supplement beads 106 may include one or more nutrients, such as vitamins, minerals, enzymes, amino acids, any other nutrient, and combinations thereof. In some embodiments, the dietary supplement beads 106 may include two or more of the nutrients. In some embodiments, the dietary supplement beads 106 may include any number of nutrients, including 1, 2, 3, 4, 5, 6, 7, 8, 9, 10, 15, 20, or more nutrients. In some embodiments, each the dietary supplement beads 106 may have the same nutrients. In some embodiments, different the dietary supplement beads 106 may have different nutrients. The dietary supplement beads 106 may release the nutrients to facilitate absorption of the nutrients in the user's body.

As discussed herein, the dietary supplement beads 106 may include a combination of nutrients. For example, the dietary supplement beads 106 may include a combination of complementary nutrients. Complementary nutrients may include nutrients that are complementary in their respective uptakes. For example, complementary nutrients may, when ingested together or when present in the user's body together, increase the uptake of one or both of the nutrients. A specific, non-limiting example of complementary nutrients includes vitamin C and iron. A specific, non-limiting example of complementary nutrients includes magnesium and zinc. A specific, non-limiting example of complementary nutrients includes magnesium and vitamin D3.

In accordance with at least one embodiment of the present disclosure, the dietary supplement beads 106 may include a combination of at least iron and vitamin C. For example, the dietary supplement beads 106 may include ingredients containing iron and/or vitamin C. These ingredients, when absorbed by the body, may maintain and/or improve the levels of the respective nutrient in the body.

As discussed herein, the user may ingest the ingestion quantity of dietary supplement beads 106. In some embodiments, the ingestion quantity of dietary supplement beads 106 may include an amount of the nutrients. In some embodiments, the ingestion quantity of the dietary supplement beads 106 may include the daily recommended value of a particular ingredient. In this manner, the user may ingest the ingestion quantity of the dietary supplement beads 106 to ingest the daily recommended value of the particular nutrient. In some embodiments, the daily recommended value may be different for different nutrients. For example, the daily recommended value in the ingestion quantity of the dietary supplement beads 106 of iron may include 8 mg for men. In some examples, the daily recommended value in the ingestion quantity of the dietary supplement beads 106 of iron may include 18 mg for women, 27 mg for pregnant women. In some examples, the daily recommended value in the ingestion quantity of the dietary supplement beads 106 of vitamin C may include 90 mg for men and 75 mg for women.

In some examples, the daily recommended value of magnesium may be 620 mg for men and 550 mg for women. In some examples, the daily recommended value of zinc may be 11 mg for men and 8 mg for women. In some examples, the daily recommended value of vitamin D3 may be 15 μg.

In some examples, the daily recommended value may be based on governmental agency data. In some examples, the daily recommended value may be based on physician input. In some examples, the daily recommended value may be based on pregnancy and/or breastfeeding status of women.

In some embodiments, the ingestion quantity of the dietary supplement beads 106 may not be greater than the daily recommended value. For example, because the vitamin C facilitates absorption of the iron, the user may absorb all of the iron in the dietary supplement beads 106. In this manner, the user may only ingest the daily recommended value of iron. In some embodiments, using the vitamin C-facilitated uptake of the iron, the user may absorb with an absorption rate 50%, 60%, 70%, 80%, 90%, 95%, 99%, 100%, or any value therebetween of the iron in the ingestion quantity of the dietary supplement beads 106. This may help to reduce the excess iron ingested by the user, which may help to reduce the undesired side-effects of excess iron ingestion. Similarly, magnesium may facilitate the absorption of zinc and/or vitamin D3 with the absorption rates described herein.

In some embodiments, the iron-including ingredient may include ferrous fumarate, ferrous sulfate, iron bisglycinate, iron gluconate, any other iron ingredient, and combinations thereof. In some embodiments, the iron-including ingredient may be a non-heme iron ingredient. Different iron-including ingredients may have different effects. For example, ferrous fumarate is an iron salt that is used as an iron supplement, which is bioavailable in the body. Iron bisglycinate is a chelated form of iron that may be more easily absorbed than some other forms of iron and may be used by individuals who have difficulty absorbing traditional forms of iron. Iron gluconate may have a mild taste, which may be usable for liquid and/or chewable supplements. In some embodiments, it may be critical that the dietary supplement beads 106 utilize iron bisglycinate to help improve iron uptake when associated with vitamin C. In some embodiments, the ingestion quantity of iron bisglycinate may be approximately 80 mg.

In some embodiments, the vitamin C-including ingredient may include ascorbic acid. Ascorbic acid is a water-soluble vitamin that is naturally found in many fruits and vegetables. Other vitamin C-including ingredients may include sodium ascorbate. In some embodiments, the vitamin C-including ingredient may include sodium ascorbate. In some embodiments, the ingestion quantity of the sodium ascorbate may be approximately 121 mg.

In some embodiments, the magnesium-including ingredient may include magnesium aspartate, magnesium citrate, magnesium lactate, magnesium chloride, magnesium L-threonate, any other magnesium-including ingredient, and combinations thereof. In some embodiments, the zinc-including ingredient may include zinc sulfate, zinc acetate, zinc gluconate, zinc picolinate, zinc glycerate, zinc citrate, zinc monomethionine, any other zinc-including ingredient, and combinations thereof.

Various nutrients in the dietary supplement beads 106 may be absorbed in the body in the small intestine 104. For example, vitamin C can improve iron absorption in the small intestine 104 by enhancing the solubility and bioavailability of non-heme iron. Iron absorption occurs in the small intestine. Heme iron is ferrous iron and is found in muscles of animals. The ferrous form is the form that can be most readily absorbed by the body. Non-heme iron is ferric iron and may be the type of iron found in plant-based foods and supplements. Non-heme iron may be less readily absorbed than heme iron.

Vitamin C can help to increase the absorption of non-heme iron by reducing ferric iron to the more easily absorbed ferrous form. Vitamin C can also chelate, or bind to, iron, which helps to prevent it from binding with other compounds in the intestine and becoming insoluble. This helps to improve the solubility and bioavailability of iron, making it more available for absorption by the body.

As a specific, non-limiting example, magnesium enhances zinc absorption through ZIP transporters, and conversely, zinc supports magnesium uptake via TRPM6 and TRPM7 channels. Through this interaction, magnesium and zinc, when bonded with glycine to form glycinate complexes, become more bioavailable. This formation not only aids in the regulation of zinc levels by magnesium but also in the facilitation of magnesium absorption by zinc, showcasing a synergistic approach to improve mineral bioavailability.

As a specific, non-limiting example, magnesium plays a critical role in the absorption and metabolism of vitamin D3 by being an essential cofactor for the enzyme 25-hydroxylase, which is pivotal in the hydroxylation of vitamin D3 to its active form, calcitriol (1,25-dihydroxyvitamin D3). This process is crucial for the biological activation of vitamin D3, facilitating its role in calcium absorption and bone health. Thus, the presence of magnesium directly enhances the efficiency of vitamin D3 metabolism, leading to improved uptake and utilization of vitamin D3.

In accordance with at least one embodiment of the present disclosure, the dietary supplement beads 106 may facilitate the simultaneous release of the complementary nutrients, such as vitamin C and iron, magnesium and zinc, and magnesium and vitamin D3, in the user's body. This may help to improve the absorption of these nutrients by the user. As a specific example, this may help to reduce the side effects of conventional iron supplements which may be caused by reduced and/or incomplete iron absorption.

In some embodiments, the dietary supplement beads 106 may include a microencapsulating material. The microencapsulating material may encapsulate both the complementary nutrients. As the microencapsulating material is dissolved in the user's body, the complementary nutrients may be released simultaneously. Simultaneous release of the complementary nutrients may help to prevent at least one of the nutrients (such as vitamin C) from being prematurely absorbed and/or excreted in the urine before the nutrients may interact to improve their relative absorption in the body.

In some embodiments, the microencapsulating material may be formed from any microencapsulating material. For example, the microencapsulating material may include gelatin, ethyl cellulose, Euhydroxypropylmethylcellulose, other cellulose derivatives, poly(lactic-co-glycolic acid), chitosan, wax, any other microencapsulating material, and combinations thereof. In some embodiments, the microencapsulating material may be selected and/or designed to dissolve and/or release the encapsulated nutrients under a specific set of conditions. For example, the microencapsulating material may be selected and/or designed to dissolve at a particular pH based on a particular portion of a user's body.

In accordance with at least one embodiment of the present disclosure, the microencapsulating material may be selected and/or designed to dissolve within the small intestine 104. In some embodiments, the microencapsulating material may be selected and/or designed to dissolve at a pH of between 5 and 6. Selecting and/or designing the microencapsulating material to dissolve at a pH of between 5 and 6, or within the small intestine, may help to release the encapsulated materials in that location. As discussed herein, the dietary supplement beads 106 may include iron and vitamin C. In some embodiments, the dietary supplement beads 106 may include a microencapsulating material that simultaneously releases the iron and the vitamin C in the small intestine 104. This may help to release the nutrients in a location where they may be absorbed. In some embodiments, the microencapsulating material may include hydroxypropylmethylcellulose, which may dissolve at a pH of between 5 and 6 within the small intestine 104. Dissolving within the small intestine 104 may help to simultaneously release the nutrients within the small intestine 104, thereby facilitating uptake of the nutrients in the small intestine 104.

In some embodiments, the dietary supplement beads 106 may facilitate the sustained release of the nutrients in the user's body. Sustained release of the nutrients in the dietary supplement beads 106 may help to improve the absorption by allowing the body to slowly take them up. Further, sustained release may help to reduce competitive absorption from other nutrients. In some embodiments, sustained release of the nutrients may help to reduce the premature absorption and/or excretion of the nutrients. For example, sustained release may help to reduce the premature absorption and/or excretion of the vitamin C before the vitamin C can facilitate absorption of the iron.

In some embodiments, the microencapsulating material may be selected and/or designed to facilitate the sustained release of the encapsulated nutrients. For example, the microencapsulating material may take time for the body to dissolve under the particular dissolution conditions. In some examples, the microencapsulating material may include hydroxypropylmethylcellulose, which may facilitate a sustained release of the iron and the vitamin C in the small intestine 104.

In some embodiments, the dietary supplement beads 106 may include a coating that may help to reduce or prevent dissolution of the dietary supplement beads 106 in the stomach 102. For example, the dietary supplement beads 106 may include an enteric coating. The enteric coating may help to protect the dietary supplement beads 106 from dissolving in the stomach 102, but may dissolve in the small intestine 104.

As discussed herein, the dietary supplement beads 106 may facilitate delayed release of two or more interfering nutrients. The interfering nutrients may interfere with one or both nutrients' uptake. Examples of interfering nutrients may include magnesium and calcium (high doses of calcium can interfere with the absorption of magnesium, leading to reduced levels of magnesium in the body), vitamin C and vitamin B12 (high doses of Vitamin C can lower the levels of intrinsic factor, which is required for the absorption of B12, lowering B12 absorption), vitamin E and vitamin K (vitamin E can interfere with the blood-clotting properties of Vitamin K. Vitamin E can also reduce the effectiveness of anticoagulant medications, calcium and iron), vitamin A and vitamin D3 (high doses of Vitamin A can interfere with the metabolism of Vitamin D, leading to reduced levels of active Vitamin D in the body), and zinc and copper (high doses of zinc can interfere with the absorption of copper, leading to reduced levels of copper in the body).

As discussed herein, in some embodiments, the dietary supplement beads 106 may include multiple different types of supplement beads, including different supplement beads having interfering nutrients. For example, the dietary supplement beads 106 may include at least two different types of supplement beads. A first supplement bead may include a first interfering nutrient and a second supplement bead may include a second interfering nutrient. The user may take both the first supplement bead and the second supplement bead such that both the first supplement bead and second supplement bead are located in the user's stomach 102 (e.g., as part of the first set of dietary supplement beads 106-1) and/or small intestine 104 at the same time (e.g., as part of the second set of dietary supplement beads 106-2).

As discussed in further detail herein, a first supplement bead may dissolve and/or release its nutrients at a first time. For example, the first supplement bead including the first interfering nutrient may dissolve and/or be at least partially processed in the user's stomach 102. The released nutrients may dissociate and travel to the small intestine 104. The released nutrients may be absorbed in the small intestine 104 as soon as they enter the small intestine 104. In some embodiments, the second supplement bead including the second interfering nutrients may be configured to dissolve in the small intestine 104. This may cause the second nutrient to be released after the second bead enters the small intestine 104. This may cause a delay in the dissociation of the second nutrient. For example, the release of the second nutrient may occur after the release of the first nutrient. In some examples, the release of the second nutrient may occur after the absorption of at least a portion of the first nutrient. In some examples, the release of the second nutrient may occur after the absorption of a majority of the first nutrient. This may help to prevent the presence of both the first nutrient and the second nutrient in the small intestine 104. This may help to improve the absorption of both the first nutrient and the second nutrient.

As a specific non-limiting example of interfering nutrients, both calcium and magnesium in the dietary supplement beads 106 may be absorbed in the body in the small intestine 104. As discussed herein, calcium and magnesium may both be absorbed at the same absorption sites in the small intestine 104. For example, calcium and magnesium may both be divalent cations and may be absorbed by the same absorption sites in the small intestine 104. This may result in competition for absorption sites, resulting in the cation having the highest concentration resulting in the highest absorption.

Further, in some situations, magnesium and calcium may both bond to certain compounds such as phytates, oxalates, and tannins present in foods to form complexes. The complexes may be insoluble in the environment in the small intestine 104. Further, the complexes may be unabsorbable at any absorption site in the small intestine. This may result in the calcium and magnesium interfering with each other's absorption. Put another way, when calcium and magnesium cations are simultaneously available in the small intestine 104, they may prevent each other from being absorbed in the small intestine 104.

In accordance with at least one embodiment of the present disclosure, and as discussed in further detail herein, to prevent the formation of the complexes and/or prevent the magnesium and calcium from interfering with absorption, the dietary supplement beads 106 may be formed to separately release the calcium and the magnesium. For example, the calcium may be released in the stomach 102 first, allowing it to be immediately available in the small intestine 104 when the calcium passes int the small intestine 104. While the calcium is being absorbed in the small intestine 104, the magnesium-including beads may be dissolving and/or released using a delayed-release mechanism in the small intestine 104. This may help to prevent or reduce the simultaneous presence of calcium cations and magnesium cations. This may reduce the interference of the calcium and the magnesium in each other's absorption.

As discussed herein, the user may ingest the ingestion quantity of dietary supplement beads 106. In some embodiments, the ingestion quantity of dietary supplement beads 106 may include an amount of the nutrients. In some embodiments, the ingestion quantity of the dietary supplement beads 106 may include the daily recommended value of a particular ingredient. In this manner, the use may ingest the ingestion quantity of the dietary supplement beads 106 to ingest the daily recommended value of the particular nutrient. In some embodiments, the daily recommended value may be different for different nutrients. For example, the daily recommended value in the ingestion quantity of the dietary supplement beads 106 of calcium may include 1,000 mg for men. In some examples, the daily recommended value in the ingestion quantity of the dietary supplement beads 106 of calcium may include 1,000 mg for women. In some examples, the daily recommended value in the ingestion quantity of the dietary supplement beads 106 of calcium may include between 400 mg and 1,500 mg. In some examples, the daily recommended value in the ingestion quantity of the dietary supplement beads 106 of magnesium may include between 30 mg and 620 mg. In some examples, the daily recommended value may be based on governmental agency data. In some examples, the daily recommended value may be based on physician input.

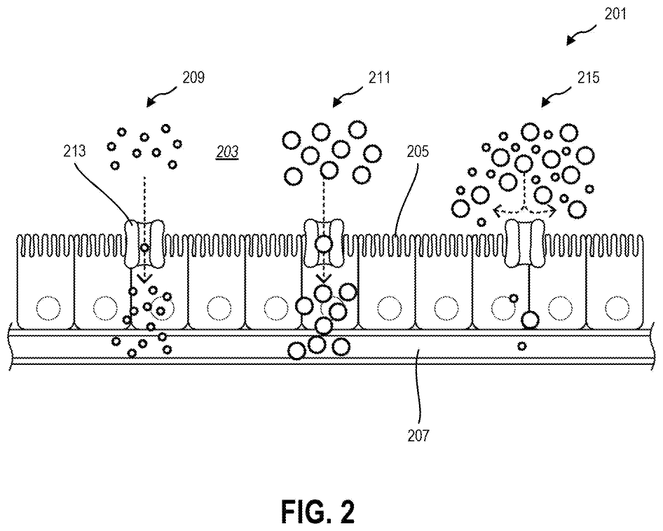

FIG. 2 is a schematic representation of small intestine 201, according to at least one embodiment of the present disclosure. The small intestine 201 includes an intestinal space 203, an epithelial wall 205, and a blood supply 207. In the illustrated embodiment, the intestinal space 203 includes calcium cations 209 and magnesium cations 211 dissolved in the intestinal space 203. As discussed herein, both the calcium cations 209 and the magnesium cations 211 may be divalent cations. For example, the calcium cations 209 may be Ca²⁺ and the magnesium cations 211 may be Mg²⁺.

The calcium cations 209 and the magnesium cations 211 may be absorbed at a divalent absorption site 213 in the epithelial wall 205. The divalent absorption site 213 may be any absorption site that may absorb divalent cations, including Ca²⁺ and Mg²⁺. Absorption of the calcium cations 209 and the magnesium cations 211 at the divalent absorption site 213 may result in the transfer of the calcium cations 209 and/or the magnesium cations 211 to the blood supply 207.

As discussed herein, when both the calcium cations 209 and the magnesium cations 211 are simultaneously present in the intestinal space 203, the calcium cations 209 and the magnesium cations 211 may compete for absorption by the same divalent absorption sites 213. This may result in a reduced absorption of the calcium cations 209 and/or the magnesium cations 211.

In some situations, when the calcium cations 209 and the magnesium cations 211 are both present in the intestinal space 203, the calcium cations 209 and the magnesium cations 211 may form a calcium-magnesium complex 215. The calcium-magnesium complex 215 may not be absorbable at the divalent absorption site 213. The calcium-magnesium complex 215 may result in the mutual interference of the calcium cations 209 and the magnesium cations 211 to absorption.

In accordance with at least one embodiment of the present disclosure, the calcium cations 209 and the magnesium cations 211 may not be present in the intestinal space 203 at the same time. for example, the calcium cations 209 may be released in the user's stomach and pass into the small intestine 201 from the stomach. This may result in the absorption of the calcium cations 209 soon after entering the intestinal space 203. While the calcium cations 209 are being absorbed, the magnesium cations 211 may be dissolved and/or released in the intestinal space 203 from a supplement bead. After the calcium cations 209 are absorbed, or at least a portion of the calcium cations 209 are absorbed, the magnesium cations 211 may be released from the magnesium-containing supplement bead, thereby freeing the magnesium cations 211 for absorption at the divalent absorption site 213 without mutual interference with the calcium cations 209.

While the embodiments of FIG. 2 have been described with respect to calcium and magnesium ions, it should be understood that the calcium cations 209 and/or the magnesium cations 211 may be replaced with other interfering cations or ions that may be absorbed at the same absorption site and compete for absorption. Examples of such competing cations and/or ions may include: zinc and copper, calcium and iron, any other competing cation pair, and combinations thereof.

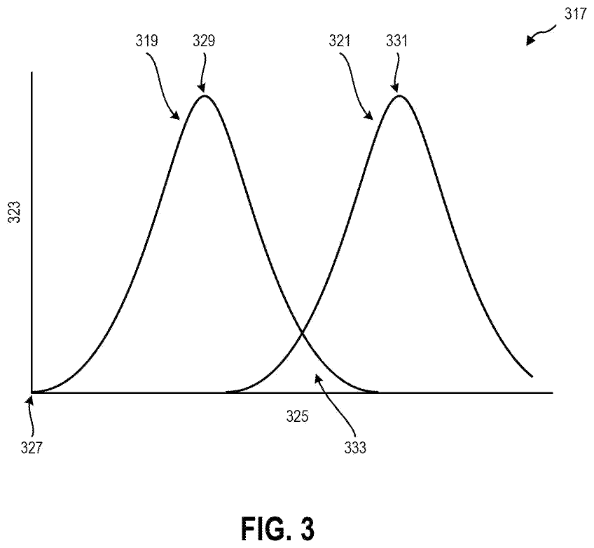

FIG. 3 is a representation of a release chart 317 for a first nutrient release line 319 and a second nutrient release line 321 with concentration 323 on the y-axis (e.g., vertical axis) and time 325 on the x-axis (e.g., horizontal axis), according to at least one embodiment of the present disclosure. In the embodiment shown, the first nutrient-containing beads and the second nutrient-containing beads may be ingested simultaneously at an ingestion time 327. As may be seen in the first nutrient release line 319, the calcium may achieve a maximum first nutrient concentration 329 in the small intestine at a first time. The first nutrient concentration may reduce over time as the first nutrient is absorbed by the small intestine.

In accordance with at least one embodiment of the present disclosure, the second nutrient may be released into the small intestine with a delayed release. This may result in an increase in the second nutrient concentration while the first nutrient concentration is reduced. For example, the second nutrient may begin to be released at a point in time after the maximum first nutrient concentration 329. This may result in a maximum second nutrient concentration 331 that occurs after the maximum first nutrient concentration 329. This may help to reduce the mutual interference in absorption of the first nutrient and the second nutrient.

In the embodiment shown, the first nutrient and the second nutrient are both present in an interference zone 333. In the interference zone, the first nutrient and the second nutrient may both be present in the small intestine. In some embodiments, the interference zone 333 may be determined based on the absorption of a percentage of the first nutrient. In some embodiments, the interference zone 333 may be determined based on the delayed release of the second nutrient into the small intestine. In some embodiments, the interference zone 333 may be minimized to reduce the amount of interference between the first nutrient and the second nutrient.

In some embodiments, the second nutrient may begin releasing after the maximum first nutrient concentration 329. In some embodiments, the maximum second nutrient concentration 331 may be after the last detectable concentration of the first nutrient is absorbed and/or processed by the body. This may help to reduce the length of the interference zone 333 and/or reduce the amount of interference between the first nutrient and the second nutrient.

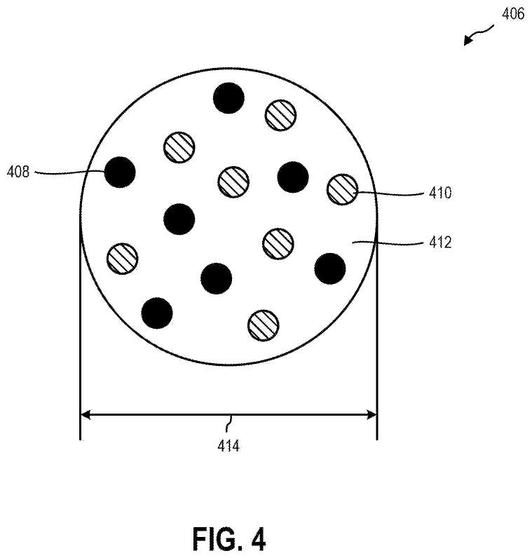

FIG. 4 is a schematic representation of a dietary supplement bead 406, according to at least one embodiment of the present disclosure. The dietary supplement bead 406 shown includes a plurality of ingredients. For example, the dietary supplement bead 406 may include a first nutrient-including ingredient 408 and a second nutrient-including ingredient 410. While the embodiments disclosed herein show two ingredients in the dietary supplement bead 406, it should be understood that the dietary supplement bead 406 of the present disclosure may include any number of ingredients, including 1, 2, 3, 4, 5, 6, 7, 8, 9, 10, or more ingredients.

When manufacturing the dietary supplement bead 406, an operator may mix the first nutrient-including ingredient 408 and the second nutrient-including ingredient 410. The mix may include other ingredients, such as other nutrients, excipients, fillers, binders, any other ingredient, and combinations thereof. The first nutrient-including ingredient 408 and/or the second nutrient-including ingredient 410 may be formed from a powder. For example, the first nutrient-including ingredient 408 and/or the second nutrient-including ingredient 410 may be formed from multiple particles. The dietary supplement bead 406 may include multiple particles of the first nutrient-including ingredient 408 and the second nutrient-including ingredient 410.

In some embodiments, the dietary supplement bead 406 may include a microencapsulating material 412. In the embodiment shown, the microencapsulating material 412 may be a binder. For example, the microencapsulating material 412 may generally fill the spaces between the particles of the first nutrient-including ingredient 408 and the second nutrient-including ingredient 410. As discussed herein, the microencapsulating material 412 may be selected and/or designed to dissolve at a particular pH or under other particular conditions. As the microencapsulating material 412 dissolves, the first nutrient-including ingredient 408 and the second nutrient-including ingredient 410 may be simultaneously released.

In the embodiment shown, the dietary supplement bead 406 is formed into a spherical particle. A spherical particle may be easy to handle and/or swallow. This may help to improve the case of use of the dietary supplement bead 406, thereby improving the compliance of the vitamin regimen.

The dietary supplement bead 406 has a bead diameter 414. In some embodiments, the bead diameter 414 may be in a range having an upper value, a lower value, or upper and lower values including any of 0.1 mm, 0.2 mm, 0.3 mm, 0.4 mm, 0.5 mm, 0.6 mm, 0.7 mm, 0.8 mm, 0.9 mm, 1.0 mm, 1.5 mm, 2.0 mm, 2.5 mm, or any value therebetween. For example, the bead diameter 414 may be greater than 0.1 mm. In another example, the bead diameter 414 may be less than 2.5 mm. In yet other examples, the bead diameter 414 may be any value in a range between 0.1 mm and 2.5 mm. In some embodiments, it may be critical that the bead diameter 414 is less than or equal to 0.5-1.0 mm to increase the case of ingestion of the dietary supplement bead 406.

In some embodiments, each of the beads in an ingestion quantity of the dietary supplement beads 406 may have the bead diameter 414. In some embodiments, manufacturing variability may result in slight differences between different beads in the ingestion quantity of the dietary supplement beads 406. In some embodiments, the bead diameter 414 may be an approximate diameter. In some embodiments, the bead diameter 414 may be an average diameter of the dietary supplement beads 406 in the ingestion quantity of the dietary supplement beads 406.

The dietary supplement bead 406 has a bead mass. In some embodiments, the bead mass may be in a range having an upper value, a lower value, or upper and lower values including any of 0.01 g, 0.05 g, 0.1 g, 0.2 g, 0.3 g, 0.4 g, 0.5 g, 0.6 g, 0.7 g, 0.8 g, 0.9 g, 1.0 g, or any value therebetween. For example, the bead mass may be greater than 0.01 g. In another example, the bead mass may be less than 1.0 g. In yet other examples, the bead mass may be any value in a range between 0.01 g and 1.0 g. In some embodiments, it may be critical that the bead mass is less than 0.5 g to increase the case of ingestion of the dietary supplement bead 406.

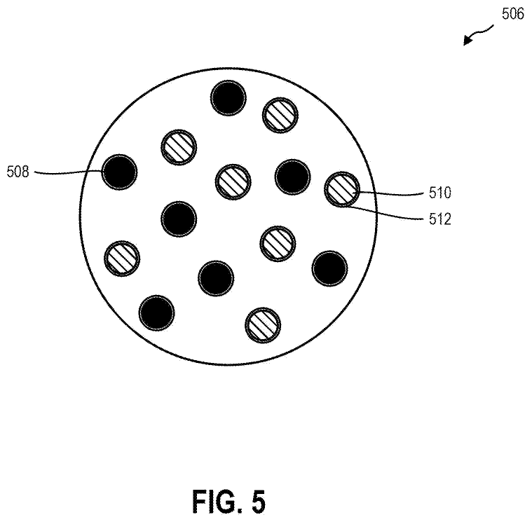

FIG. 5 is a schematic representation of a dietary supplement bead 506, according to at least one embodiment of the present disclosure. The dietary supplement bead 506 shown includes a plurality of ingredients. For example, the dietary supplement bead 506 may include a first nutrient-including ingredient 508 and a second nutrient-including ingredient 510. The dietary supplement bead 506 may include any of the properties discussed above with respect to FIG. 4.

In the embodiment shown, the nutrient particles of the dietary supplement bead 506 may include a microencapsulating material 512 that forms a coating around the individual grains of the first nutrient-including ingredient 508 and the second nutrient-including ingredient 510. The microencapsulating material 512 forming a coating over the first nutrient-including ingredient 508 and the second nutrient-including ingredient 510 may help to facilitate release of the first nutrient and the second nutrient at the particular time and/or in a particular location.

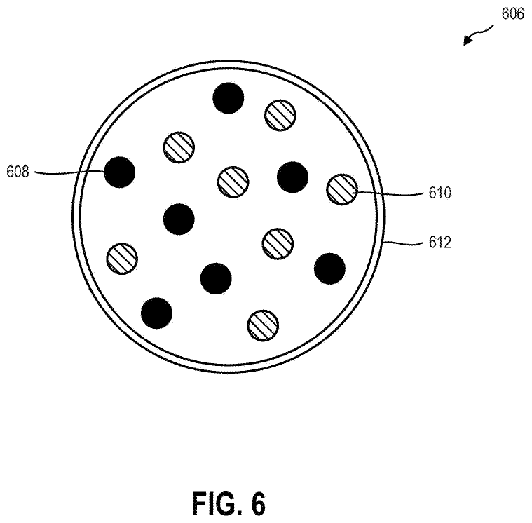

FIG. 6 is a schematic representation of a dietary supplement bead 606, according to at least one embodiment of the present disclosure. The dietary supplement bead 606 shown includes a plurality of ingredients. For example, the dietary supplement bead 606 may include a first nutrient-including ingredient 608 and a second nutrient-including ingredient 610. The dietary supplement bead 606 may include any of the properties discussed above with respect to FIG. 4 and/or FIG. 5.

In the embodiment shown, the nutrient particles of the dietary supplement bead 606 may include a microencapsulating material 612 that forms a coating around an exterior of the dietary supplement bead 606. This may help to control the release of the first nutrient-including ingredient 608 and/or the second nutrient-including ingredient 610 to a particular location and/or particular conditions that may dissolve the microencapsulating material 612. For example, the microencapsulating material 612 may include an enteric coating that may help to reduce or prevent dissolution of the dietary supplement bead 606 in the stomach of the user, thereby allow for the release of the first nutrient-including ingredient 608 and/or second nutrient-including ingredient 610 in the small intestine of the user.

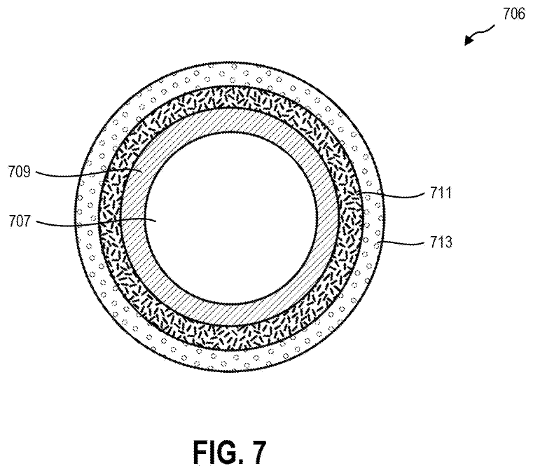

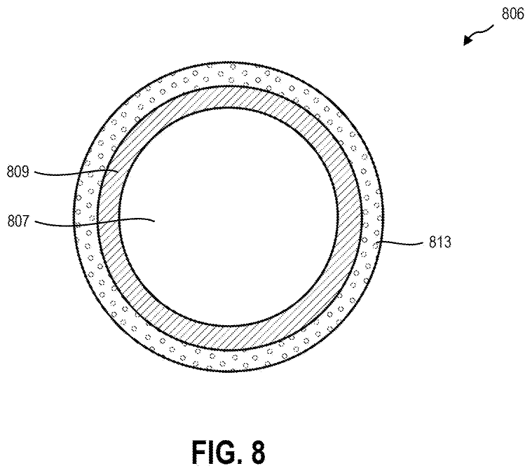

FIG. 7 is a schematic representation of a dietary supplement bead 706, according to at least one embodiment of the present disclosure. The dietary supplement bead 706 shown includes a layers built surrounding a body 707. The body 707 may include one or more nutrient-including ingredients, as discussed herein. The dietary supplement bead 706 may include any of the properties discussed above with respect to FIG. 4, FIG. 5, and/or FIG. 6.

A first layer surrounding the body 707 may be a sustained-release coating 709. The sustained-release coating 709 may be configured to slow the release of the nutrients from the body 707 over a period of time (as discussed above with respect to FIG. 3). Surrounding the sustained-release coating 709 may be a delayed release coating 711. The delayed release coating 711 may delay the release of the nutrients from the body 707. For example, the delayed release coating 711 may delay the release of the nutrients while the dietary supplement bead 706 is located within the user's intestines. Surrounding the delayed release coating 711 may be an enteric coating 713. The enteric coating 713 may facilitate dissolution of the dietary supplement bead 706 within the intestine of the user.

As may be seen, the release profile may utilize a combination of two or more of the sustained-release coating 709, delayed release coating 711, and enteric coating 713 using a blend of hydroxypropyl methylcellulose (HPMC), ethylcellulose (EC), cornstarch, Eudragit and microcrystalline cellulose (MCC). Eudragit, HPMC and EC, are polymers used in pharmaceutical formulations to control the release of active ingredients. HPMC is a water-soluble polymer that forms a gel-like layer around the supplement particles, while EC is a water-insoluble polymer that forms a barrier to protect the supplement from stomach acid. By combining HPMC and EC, the dietary supplement bead 706 exhibits a delayed-release effect that slowly releases the supplement over time rather than all at once.

For example, the HPMC layer (e.g., the enteric coating 713) initially swells in the stomach, forming a barrier that delays the release of the supplement. As the HPMC gradually dissolves, the EC layer (e.g., the delayed release coating 711) takes over to provide further protection and delay the release even more. The dietary supplement bead 706 may utilize cornstarch as a binder to hold the supplement particles together and MCC as a filler to add bulk to the formulation. Together, these ingredients create an enteric coating that protects the supplement from stomach acid and delays its release until it reaches the small intestine, where it can be more effectively absorbed by the body. Overall, the blend of HPMC, EC, cornstarch, and MCC creates a sustained-release, delayed-release, and enteric-coated supplement that is protected from the harsh environment of the stomach and gradually released over time for improved absorption by the body.

FIG. 8 is a schematic representation of a dietary supplement bead 806, according to at least one embodiment of the present disclosure. The dietary supplement bead 806 shown includes layers built surrounding a body 807. The body 807 may include one or more nutrient-including ingredients, as discussed herein. The dietary supplement bead 806 may include any of the properties discussed above with respect to FIG. 4, FIG. 5, and/or FIG. 6.

A first layer surrounding the body 807 may be a sustained-release coating 809. The sustained-release coating 809 may be configured to slow the release of the nutrients from the body 807 over a period of time (as discussed above with respect to FIG. 3). Surrounding the sustained-release coating 809 may be an enteric coating 813. The enteric coating 813 may facilitate dissolution of the dietary supplement bead 806 within the intestine of the user.

As may be seen in a comparison between the dietary supplement bead 706 of FIG. 8 and the dietary supplement bead 806 of FIG. 8, the dietary supplement bead 806 may not include the delayed release coating. In this manner, the dietary supplement bead 706 may include a first nutrient and the dietary supplement bead 806 may include a second nutrient that may interfere with absorption of the first nutrient. By taking the dietary supplement bead 806 and the dietary supplement bead 706 simultaneously, the interfering nutrients may be released (and therefore absorbed) at different times, thereby improving the overall uptake of the nutrients.

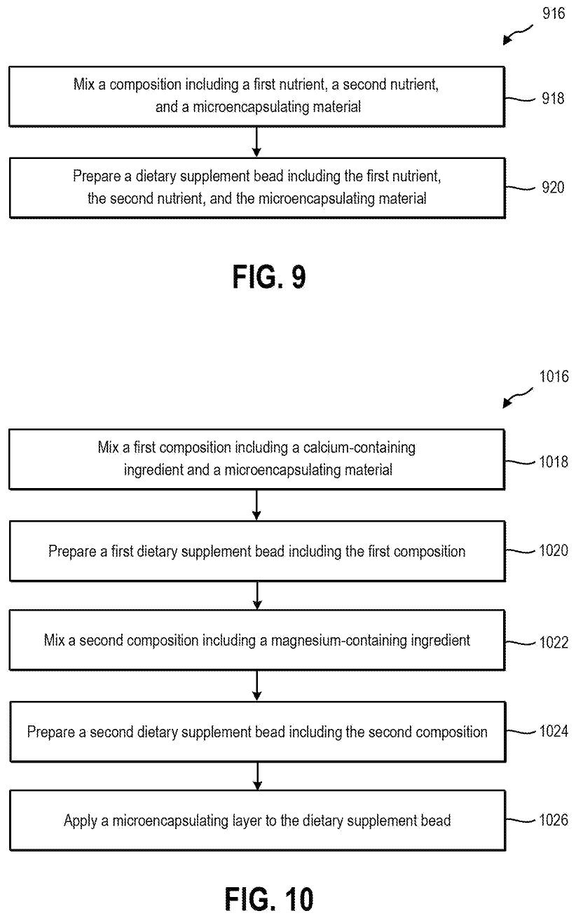

FIG. 9 is a representation of a method 916 for manufacturing a dietary supplement bead, according to at least one embodiment of the present disclosure. The method 916 may include mixing a composition including a first nutrient, a second nutrient, and a microencapsulating material at 918. The microencapsulating material may be selected and/or designed to facilitate the release of the first nutrient and the second nutrient in the small intestine of the user. In some embodiments, the microencapsulating material may be selected or designed to dissolve in the small intestine of the user. The first nutrient and the second nutrient may be complementary nutrients. In this manner, the first nutrient and the second nutrient may be simultaneously released to improve the uptake of one or both of the first nutrient and the second nutrient.

In some embodiments, mixing the composition may include preparing a powder of the first nutrient-including ingredient and the second nutrient-including ingredient. In some embodiments, the powder may be mixed to evenly distribute the first nutrient-including ingredient and the second nutrient-including ingredient. In some embodiments, the microencapsulating material may bind the powder together.

In some embodiments, the method 916 may include preparing a dietary supplement bead including the first nutrient, the second nutrient, and the microencapsulating material. In some embodiments, preparing the dietary supplement bead may include cutting or otherwise separating the bound powder into the beads having the bead diameter and bead mass.

In some embodiments, beads containing the active ingredients are prepared using extrusion and spheronization, spray drying, and fluid bed granulation. There are two common methods for applying coatings to beads: fluid bed coating and pan coating.

Fluid bed coaters suspend the beads in a stream of air, creating a fluidized bed, while the coating solution is sprayed onto the beads. The coating solution is atomized using a spray nozzle, and the solvent is evaporated using heated air. Fluid bed coaters are versatile and can be used for all three coating types: enteric, delayed-release, and sustained-release.

In pan coating, beads are placed in a rotating pan or drum, and the coating solution is sprayed onto the beads as they tumble. Pan coaters can be equipped with spray nozzles, air handling systems, and temperature controls for precise application of the coatings. Pan coating is suitable for enteric and sustained-release coatings but may be less suitable for some delayed-release coatings, depending on the specific formulation.

The coating process may involve applying the coatings sequentially, starting with the sustained release coating, followed by the delayed release coating then lastly, the exterior layer is enterically coated. Each layer may be dried before applying the next one to ensure proper separation and functionality of the coatings.

A coating solution containing the sustained-release formulation e.g., ethylcellulose (EC), is prepared and sprayed onto the delayed-release coated beads using fluid bed or pan coating equipment. The coated beads are then dried to remove the solvent.

A coating solution containing the delayed-release formulation (e.g., HPMC) is prepared and sprayed onto the enteric-coated beads using fluid bed equipment. The coated beads are then dried to remove the solvent.

A coating solution containing an enteric polymer (e.g., Eudragit S100) is prepared and sprayed onto the beads using either fluid bed or pan coating equipment. The coated beads are then dried to remove the solvent.

As may be understood, the method 916 may be used to generate any type of bead, including beads including complementary nutrients, and different beads including interfering nutrients. For example, FIG. 10 illustrates a method 1016 for manufacturing a plurality of dietary supplement beads, according to at least one embodiment of the present disclosure. A manufacturer may mix a first composition including a first nutrient-containing ingredient and a microencapsulating material at 1018. The microencapsulating material may be configured to release the first nutrient-containing ingredient in a first time (such as at a stomach of the user). The manufacturer may prepare a first dietary supplement bead including the first composition at 1020. In some embodiments, the microencapsulating material may be an excipient or a binder and may not include any delayed and/or sustained release materials.

The manufacturer may mix a second composition including a second nutrient-containing ingredient at 1022. The manufacturer may prepare a second dietary supplement bead including the second nutrient-containing ingredient at 1024. The manufacturer may apply a microencapsulating layer to the dietary supplement bead at 1026. The microencapsulating layer may be configured to release the first nutrient-containing ingredient at a second time (e.g., in a small intestine of the user). As discussed herein, in some embodiments, the first dietary supplement bead and the second dietary supplement bead may be configured to be taken simultaneously with each other.

As discussed above with respect to FIG. 9, the first bead and the second bead may be manufactured in any manner to facilitate the formation of the various sustained release layers and/or delayed release layers. Indeed, the first bead may have a different combination of layers than the second layer. This may facilitate the release of interfering nutrients at different times.

In some embodiments, the first bead may include nutrients that are complementary with at least one other nutrient in the first bead. In some embodiments, every nutrient in the first bead may be complementary with every other nutrient in the first bead. In some embodiments, a nutrient may be complementary with a single other nutrient in the first bead. In some embodiments, a nutrient may be complementary with a plurality of other nutrients in the first bead.

In some embodiments, the second bead may include nutrients that are complementary with at least one other nutrient in the second bead. In some embodiments, every nutrient in the second bead may be complementary with every other nutrient in the second bead. In some embodiments, a nutrient may be complementary with a single other nutrient in the second bead. In some embodiments, a nutrient may be complementary with a plurality of other nutrients in the second bead.

In some embodiments, the first bead and the second bead may include complementary nutrients. For example, the first bead and the second bead may include the same or a similar release profile and nutrients from the first bead and the second bead may be complementary.

In some embodiments, as discussed herein, the first bead may include complementary nutrients to other nutrients in the first bead, and the second bead may include complementary nutrients to other nutrients in the second bead, and the first bead and the second bead may include interfering nutrients. In this manner, the bead set may include both complementary nutrients and interfering nutrients that may be ingested simultaneously.

### INDUSTRIAL APPLICABILITY

Following are sections in accordance with at least one embodiment of the present disclosure:
A1. A dietary supplement bead, comprising:
   a body comprising iron and vitamin C; and
   a microencapsulating material in the body, the microencapsulating material facilitating a sustained release of the iron and the vitamin C simultaneously within an intestine of a user.
A2. The dietary supplement bead of section A1, wherein the body is spherical.
A3. The dietary supplement bead of any of sections A1 or A2, wherein the microencapsulating material is configured to not release any of the vitamin C or the iron in a stomach of the user.
A4. The dietary supplement bead of any of sections A1-A3, wherein the microencapsulating material includes hydroxypropylmethylcellulose.
A5. The dietary supplement bead of any of sections A1-A4, wherein the microencapsulating material dissolves with a pH of between 5 and 6.
A6. The dietary supplement bead of any of sections A1-A5, wherein the body has a diameter of approximately 1.0 mm.
A7. The dietary supplement bead of any of sections A1-A6, wherein the body and the microencapsulating material have a mass of approximately 0.5 g.
A8. The dietary supplement bead of any of sections A1-A7, wherein the vitamin C includes sodium ascorbate.
A9. The dietary supplement bead of any of sections A1-A8, wherein the iron includes iron bisglycinate.
B1. A dietary supplement, comprising:
   a plurality of dietary supplement beads, each bead of the plurality of dietary supplement beads including:
      a body comprising iron and vitamin C, wherein the plurality of dietary supplement beads includes an iron daily recommended value of iron and a vitamin C daily recommended value of vitamin C; and
      a microencapsulating material in the body to facilitate a sustained release of the iron and vitamin C simultaneously.
B2. The dietary supplement of section B1, wherein the plurality of dietary supplement beads has a total weight of approximately 4.8 g.
B3. The dietary supplement of any of sections B1 or B2, wherein the vitamin C includes sodium ascorbate and wherein the plurality of dietary supplement beads include approximately 121 mg of the sodium ascorbate.
B4. The dietary supplement of any of sections B1-B3, wherein the iron includes ferrous bisglycinate and wherein the plurality of dietary supplement beads include approximately 80 mg of the ferrous bisglycinate.
B5. The dietary supplement of any of sections B1-B4, wherein a quantity of the plurality of dietary supplement beads is approximately 10.
B6. The dietary supplement of any of sections B1-B5, wherein the microencapsulating material dissolves at a pH of between 5 and 6.
B7. The dietary supplement of any of sections B1-B6, wherein the iron and vitamin C have an absorption rate of approximately 100%.
B8. The dietary supplement of any of sections B1-B7, wherein each of plurality of dietary supplement beads have an average diameter of approximately 1.0 mm.
C1. A method for manufacturing a dietary supplement, comprising:
   mixing a composition including iron, vitamin C, and a microencapsulating material, the microencapsulating material facilitating release the iron and the vitamin C simultaneously in a small intestine of a user; and
   preparing a dietary supplement bead including the iron, the vitamin C, and the microencapsulating material.
C2. The method of section C1, wherein preparing the dietary supplement bead includes preparing a plurality of beads having a diameter of approximately 1.0 mm.
C3. The method of section C2, wherein the plurality of beads include an iron daily recommended value of the iron and a vitamin C daily recommended value of the vitamin C.
D1. A dietary supplement bead set to be swallowed simultaneously by a user, the dietary supplement bead set comprising:
   a first dietary supplement bead, the first dietary supplement bead including:
      a first body including a calcium-containing ingredient; and
      a microencapsulating material in the body, the microencapsulating material facilitating a release of the calcium-containing ingredient in a stomach of said user; and
   a second dietary supplement bead, the second dietary supplement bead including: a second body including a magnesium-containing ingredient; and
      a microencapsulating layer surrounding the body, the microencapsulating layer facilitating a release of the magnesium-containing ingredient in a small intestine of said user.
D2. The dietary supplement bead of section D1, wherein the body is spherical.
D3. The dietary supplement bead of any of sections D1 or D2, wherein the microencapsulating material is configured to not release any of the magnesium-containing ingredient in a stomach of the user.
D4. The dietary supplement bead of any of sections D1-D3, wherein the microencapsulating material includes hydroxypropylmethylcellulose.
D5. The dietary supplement bead of any of sections D1-D4, wherein the microencapsulating material dissolves with a pH of between 5 and 6.
D6. The dietary supplement bead of any of sections D1-D5, wherein the body has a diameter of approximately 1.0 mm.
D7. The dietary supplement bead of any of sections D1-D6, wherein the body and the microencapsulating material have a mass of approximately 0.5 g.
D8. The dietary supplement bead of any of sections D1-D7, wherein the calcium-containing ingredient includes calcium-carbonate.
D9. The dietary supplement bead of any of sections D1-D8, wherein the magnesium-containing ingredient includes magnesium citrate.
E1. A dietary supplement, comprising:
   a plurality of dietary supplement beads including a first set of dietary supplement beads and a second set of dietary supplement beads, each first dietary supplement bead of the first set of dietary supplement beads including a body including a calcium-containing containing ingredient, the body configured to release the calcium-containing ingredient in a stomach of a user, wherein the first set of dietary supplement beads includes a calcium daily recommended value of calcium, and wherein each second dietary supplement bead of the second set of dietary supplement beads includes:
      a second body including a magnesium-containing ingredient, wherein the second set of dietary supplement beads includes a magnesium daily recommended value of magnesium; and
      a microencapsulating layer surrounding the body, the microencapsulating layer facilitating a release of the magnesium-containing ingredient in a small intestine of said user.
E2. The dietary supplement of section E1, wherein the plurality of dietary supplement beads has a total weight of approximately 5.5 g.
E3. The dietary supplement of any of sections E1 or E2, wherein at least one first bead from the first set of dietary supplement beads and at least one second bead from the second set of dietary supplement beads are configured to be taken simultaneously.
E4. The dietary supplement of section E3, wherein the at least one first bead and the at least one second bead are configured to be taken within 10 minutes of each other.
E5. The dietary supplement of any of sections E1-E4, wherein a quantity of the plurality of dietary supplement beads is approximately 10.
E6. The dietary supplement of any of sections E1-E5, wherein the microencapsulating material dissolves at a pH of between 5 and 6.
E7. The dietary supplement of any of sections E1-E6, wherein the calcium-containing ingredient and magnesium-containing ingredient have an absorption rate of approximately 100%.
E8. The dietary supplement of any of sections E1-E7, wherein each of plurality of dietary supplement beads have an average diameter of approximately 1.0 mm.
F1. A method for manufacturing a dietary supplement, comprising:
   mixing a first composition including a calcium-containing ingredient and a microencapsulating material, the microencapsulating material facilitating release of the calcium-containing ingredient in a stomach of a user; and
   preparing a first dietary supplement bead including the first composition;
   mixing a second composition including a magnesium-containing ingredient;
   preparing a second dietary supplement bead including the second composition; and
   applying a microencapsulating layer to the dietary supplement bead, the microencapsulating layer facilitating release of the magnesium-containing ingredient in a small intestine of said user, wherein the first dietary supplement bead and the second dietary supplement bead are configured to be taken simultaneously.
F2. The method of section F1, wherein preparing the first dietary supplement bead and the second dietary supplement bead includes preparing a plurality of beads having a diameter of approximately 1.0 mm.
F3. The method of any of sections F1 or F2, further comprising instructing said user to swallow the first dietary supplement bead and the second dietary supplement bead simultaneously.
G1. A dietary supplement bead, comprising:
   a body including a first nutrient and a second nutrient, the first nutrient and the second nutrient facilitating complementary uptake of a user; AND
   a microencapsulating material in the body, the microencapsulating material facilitating a sustained release of the first nutrient and the second nutrient simultaneously within an intestine of a user.
G2. The dietary supplement bead of section G1, wherein the first nutrient includes at least one of vitamin C or magnesium and the second nutrient includes at least one of iron, zinc, or Vitamin D3.
G3. The dietary supplement bead of section G2, wherein the first nutrient includes vitamin C and magnesium and the second nutrient includes iron, zinc, and vitamin D3.
G4. The dietary supplement bead of any of sections G1-G3, wherein the microencapsulating material dissolves with a pH of between 5 and 6.
G5. The dietary supplement bead of any of sections G1-G4, wherein the body has a diameter of approximately 1.0 mm.
G6. The dietary supplement bead of any of sections G1-G5, wherein the body and the microencapsulating material have a mass of approximately 0.5 g.
H1. A dietary supplement bead set to be swallowed simultaneously by a user, the dietary supplement bead set comprising:
   a first dietary supplement bead, the first dietary supplement bead including: a first body including a first nutrient; and
      a microencapsulating material in the body, the microencapsulating material facilitating a release of the first nutrient at a first time; and
   a second dietary supplement bead, the second dietary supplement bead including: a second body including a second nutrient; and
      a microencapsulating layer surrounding the body, the microencapsulating layer facilitating a release of the second nutrient at a second time.
H2. The dietary supplement bead set of section H1, wherein the first nutrient includes at least one of vitamin C, vitamin E, iron, vitamin D3, calcium, and zinc, and wherein the second nutrient includes at least one of iron, magnesium, vitamin B12, vitamin K, vitamin A, and copper.
H3. The dietary supplement bead set of any of sections H1 or H2, wherein the first dietary supplement bead includes a first dietary supplement bead subset, the first dietary supplement bead subset including:
   a first bead including the vitamin C;
   a second bead including the vitamin E;
   a third bead including the iron;
   a fourth bead including the vitamin D3;
   a fifth bead including the calcium; and
   a sixth bead including the zinc; and
   and wherein the second dietary supplement bead includes a second dietary supplement bead subset, the second dietary bead subset including:
      a first bead including the iron;
      a second bead including the magnesium;
      a third bead including the vitamin B12;
      a fourth bead including the vitamin K;
      a fifth bead including the vitamin A; and
      a sixth bead including the copper.
H4. The dietary supplement bead set of any of sections H1-H3, wherein the microencapsulating material dissolves with a pH of between 5 and 6.
H5. The dietary supplement bead set of any of sections H1-H4, wherein the body has a diameter of approximately 1.0 mm.
H6. The dietary supplement bead set of any of sections H1-H5, wherein the body and the microencapsulating material have a mass of approximately 0.5 g.
I1. A dietary supplement bead set, comprising:
   a first dietary supplement bead including:
      a first body including a first nutrient and a second nutrient; and
      a first microencapsulating layer surrounding the first body, the first microencapsulating layer facilitating a sustained release of the first nutrient and the second nutrient simultaneously at a first time; and
   a second dietary supplement bead including:
      a second body including a third nutrient; and
      a second microencapsulating layer surrounding the second body, the second microencapsulating material layer a release of the third material at a second time different than the first time.

I2. The dietary supplement bead set of section I1, wherein the first nutrient includes vitamin C, the second nutrient includes iron, and the third nutrient includes calcium.

I3. The dietary supplement bead set of any of sections I1 or I2, wherein the first nutrient includes vitamin C, the second nutrient includes iron, and the third nutrient includes vitamin B12.

I4. The dietary supplement bead set of any of sections I1-I3, wherein the first nutrient includes magnesium, the second nutrient includes zinc, and the third nutrient includes calcium.

I5. The dietary supplement bead set of any of sections I1-I4, wherein the first nutrient includes magnesium, the second nutrient includes zinc, and the third nutrient includes copper.

I6 The dietary supplement bead set of any of sections I1-I5, wherein the first nutrient includes magnesium, the second nutrient includes vitamin D3, and the third nutrient includes calcium.
I7. The dietary supplement bead set of any of sections I1-I6, wherein the first nutrient includes magnesium, the second nutrient includes vitamin D3, and the third nutrient includes vitamin A.

I8. The dietary supplement bead set of any of sections I1-I7, wherein the first body and the second body have a diameter of approximately 1.0 mm and a mass of approximately 0.5 g.

One or more specific embodiments of the present disclosure are described herein. These described embodiments are examples of the presently disclosed techniques. Additionally, in an effort to provide a concise description of these embodiments, not all features of an actual embodiment may be described in the specification. It should be appreciated that in the development of any such actual implementation, as in any engineering or design project, numerous embodiment-specific decisions will be made to achieve the developers' specific goals, such as compliance with system-related and business-related constraints, which may vary from one embodiment to another. Moreover, it should be appreciated that such a development effort might be complex and time consuming, but would nevertheless be a routine undertaking of design, fabrication, and manufacture for those of ordinary skill having the benefit of this disclosure.

The articles “a,” “an,” and “the” are intended to mean that there are one or more of the elements in the preceding descriptions. The terms “comprising,” “including,” and “having” are intended to be inclusive and mean that there may be additional elements other than the listed elements. Additionally, it should be understood that references to “one embodiment” or “an embodiment” of the present disclosure are not intended to be interpreted as excluding the existence of additional embodiments that also incorporate the recited features. For example, any element described in relation to an embodiment herein may be combinable with any element of any other embodiment described herein. Numbers, percentages, ratios, or other values stated herein are intended to include that value, and also other values that are “about” or “approximately” the stated value, as would be appreciated by one of ordinary skill in the art encompassed by embodiments of the present disclosure. A stated value should therefore be interpreted broadly enough to encompass values that are at least close enough to the stated value to perform a desired function or achieve a desired result. The stated values include at least the variation to be expected in a suitable manufacturing or production process, and may include values that are within 5%, within 1%, within 0.1%, or within 0.01% of a stated value.

A person having ordinary skill in the art should realize in view of the present disclosure that equivalent constructions do not depart from the spirit and scope of the present disclosure, and that various changes, substitutions, and alterations may be made to embodiments disclosed herein without departing from the spirit and scope of the present disclosure. Equivalent constructions, including functional “means-plus-function” clauses are intended to cover the structures described herein as performing the recited function, including both structural equivalents that operate in the same manner, and equivalent structures that provide the same function. It is the express intention of the applicant not to invoke means-plus-function or other functional claiming for any claim except for those in which the words ‘means for’ appear together with an associated function. Each addition, deletion, and modification to the embodiments that falls within the meaning and scope of the claims is to be embraced by the claims.

The terms “approximately,” “about,” and “substantially” as used herein represent an amount close to the stated amount that still performs a desired function or achieves a desired result. For example, the terms “approximately,” “about,” and “substantially” may refer to an amount that is within less than 5% of, within less than 1% of, within less than 0.1% of, and within less than 0.01% of a stated amount. Further, it should be understood that any directions or reference frames in the preceding description are merely relative directions or movements. For example, any references to “up” and “down” or “above” or “below” are merely descriptive of the relative position or movement of the related elements.

The present disclosure may be embodied in other specific forms without departing from its spirit or characteristics. The described embodiments are to be considered as illustrative and not restrictive. The scope of the disclosure is, therefore, indicated by the appended claims rather than by the foregoing description. Changes that come within the meaning and range of equivalency of the claims are to be embraced within their scope.

What is claimed is:

1. A dietary supplement bead, comprising:
a body including a first nutrient and a second nutrient, the first nutrient and the second nutrient facilitating complementary uptake of a user; AND
a microencapsulating material in the body, the microencapsulating material facilitating a sustained release of the first nutrient and the second nutrient simultaneously within an intestine of a user.

2. The dietary supplement bead of claim 1, wherein the first nutrient includes at least one of vitamin C or magnesium and the second nutrient includes at least one of iron, zinc, or Vitamin D3.

3. The dietary supplement bead of claim 2, wherein the first nutrient includes vitamin C and magnesium and the second nutrient includes iron, zinc, and vitamin D3.

4. The dietary supplement bead of claim 1, wherein the microencapsulating material dissolves with a pH of between 5 and 6.

5. The dietary supplement bead of claim 1, wherein the body has a diameter of approximately 1.0 mm.

6. The dietary supplement bead of claim 1, wherein the body and the microencapsulating material have a mass of approximately 0.5 g.

7. A dietary supplement bead set to be swallowed simultaneously by a user, the dietary supplement bead set comprising:
a first dietary supplement bead, the first dietary supplement bead including:
a first body including a first nutrient; and
a first microencapsulating layer in the body, the first microencapsulating layer facilitating a release of the first nutrient at a first time; and
a second dietary supplement bead, the second dietary supplement bead including:
a second body including a second nutrient; and
a second microencapsulating layer surrounding the body, the microencapsulating layer facilitating a release of the second nutrient at a second time.

8. The dietary supplement bead set of claim 7, wherein the first nutrient includes at least one of vitamin C, vitamin E, iron, vitamin D3, calcium, and zinc, and wherein the second nutrient includes at least one of iron, magnesium, vitamin B12, vitamin K, vitamin A, and copper.

9. The dietary supplement bead set of claim 7, wherein the first dietary supplement bead includes a first dietary supplement bead subset, the first dietary supplement bead subset including:
a first bead including the vitamin C;
a second bead including the vitamin E;
a third bead including the iron;
a fourth bead including the vitamin D3;
a fifth bead including the calcium; and
a sixth bead including the zinc; and
and wherein the second dietary supplement bead includes a second dietary supplement bead subset, the second dietary bead subset including:
a first bead including the iron;
a second bead including the magnesium;
a third bead including the vitamin B12;
a fourth bead including the vitamin K;
a fifth bead including the vitamin A; and
a sixth bead including the copper.

10. The dietary supplement bead set of claim 7, wherein the microencapsulating material dissolves with a pH of between 5 and 6.

11. The dietary supplement bead set of claim 7, wherein the body has a diameter of approximately 1.0 mm.

12. The dietary supplement bead set of claim 7, wherein the body and the microencapsulating material have a mass of approximately 0.5 g.

13. A dietary supplement bead set, comprising:
a first dietary supplement bead including:
a first body including a first nutrient and a second nutrient; and
a first microencapsulating layer surrounding the first body, the first microencapsulating layer facilitating a sustained release of the first nutrient and the second nutrient simultaneously at a first time; and
a second dietary supplement bead including:
a second body including a third nutrient; and
a second microencapsulating layer surrounding the second body, the second microencapsulating material layer facilitating a release of the third material at a second time different than the first time.

14. The dietary supplement bead set of claim 13, wherein the first nutrient includes vitamin C, the second nutrient includes iron, and the third nutrient includes calcium.

15. The dietary supplement bead set of claim 13, wherein the first nutrient includes vitamin C, the second nutrient includes iron, and the third nutrient includes vitamin B12.

16. The dietary supplement bead set of claim 13, wherein the first nutrient includes magnesium, the second nutrient includes zinc, and the third nutrient includes calcium.

17. The dietary supplement bead set of claim 13, wherein the first nutrient includes magnesium, the second nutrient includes zinc, and the third nutrient includes copper.

18. The dietary supplement bead set of claim 13, wherein the first nutrient includes magnesium, the second nutrient includes vitamin D3, and the third nutrient includes calcium.

19. The dietary supplement bead set of claim 13, wherein the first nutrient includes magnesium, the second nutrient includes vitamin D3, and the third nutrient includes vitamin A.

20. The dietary supplement bead set of claim 13, wherein the first body and the second body have a diameter of approximately 1.0 mm and a mass of approximately 0.5 g.
